(19) United States
(12) Patent Application Publication
(10) Pub. No.: US 2008/0160583 A1
(43) Pub. Date: Jul. 3, 2008

(54) Process for co-fermentation of phellinus linteus fungus with chinese herbal medicines

(75) Inventors: Kuo-Chuan Wang, Yong Kang City (TW); Chia-Hsin Huang, Yong Kang City (TW)

Correspondence Address: KAMRATH & ASSOCIATES P.A., 4825 OLSON MEMORIAL HIGHWAY, SUITE 245, GOLDEN VALLEY, MN, 55422, US

(73) Assignee: I CHUAN BIO-TECH CORP., Yong Kang City (TW)

(21) Appl. No.: 11/649,023

(22) Filed: Jan. 3, 2007

(51) Int. Cl. C12P 19/04

(52) U.S. Cl. 435/101

(57) ABSTRACT

The present invention relates to a process of fermenting a strain of Phellinus linteus fungus, in which the fungus is incubated in a medium under the condition for the best yield control with the aim to obtain the best yield of mycelia and polysaccharides, where the best ratio of the medium having potato extract and glucose contains 200 g of potato extract and 20 g glucose per liter. The fungus is inoculated to the medium and can generate the best yield of mycelia and polysaccharides under the condition for the best yield control. Moreover, the co-fermentation of the Phellinus linteus fungus with a single- or multiple-component formulation of Chinese herbal medicines (more than two different kinds of Chinese herbal medicine) is shown to generate a better yield of mycelia and polysaccharides as well as an increase in the anti-tumor activity, compared to the fermentation of the Phellinus linteus fungus alone.

### FIELD OF THE INVENTION

The present invention relates to a process of fermenting the fungus of the Phellinus linteus which utilizes a liquid medium and generates the best yield of mycelia and polysaccharides under the condition for the best yield. Moreover, the co-fermentation of the Phellinus linteus fungus with a single- or multiple-component formulation of Chinese herbal medicines (one kind or more than two different kinds of Chinese herbal medicines) is shown to generate a better yield of mycelia and polysaccharides as well as an increase in the anti-tumor activity, compared to the fermentation of the Phellinus linteus fungus alone.

### DESCRIPTION OF THE PRIOR ART

Phellinus linteus is a mushroom found on wild mulberry trees, which is black in color, lump-shape, hard and bright-yellow inside. It is therefore called mulberry yellow. As in the BenCao Gangmu (Materia Medica by Li Shizhen) recorded, the Phellinus linteus is cold in nature, bitter for taste, beneficial for the five organs (heart, liver, spleen, lung and kidney) and can eliminate poisonous materials and stop bleeding. In Korea and Japan, the Phellinus linteus fungus is shown to have the effects such as abdominal pain relief, diuresis, antitumor, stomach strengthening and antidiarrhea. The studies in Korea and Japan have demonstrated that polysaccharides isolated from Phellinus linteus markedly stimulate the immune response of the T-cells. The inhibitory effects of Phellinus linteus polysaccharides on tumor growth and metastasis are achieved by activation of the immune system.

### SUMMARY OF THE INVENTION

Since the Phellinus linteus extract imported from Japan is too costly and the efficacy and purity thereof are mostly uncertain, it is an object of the present invention to provide a process of fermenting the Phellinus linteus fungus in a liquid medium, where the fungus is grown under the best condition of carbon source, nitrogen source, inorganic salts, pH value and rotation speed with the aim to obtain a better yield of mycelia and polysaccharides of the Phellinus linteus. The object is solved according to the features described in the claim 1. Additionally, it is another object of the present invention to provide a process of co-fermenting the Phellinus linteus fungus with a single- or multiple-component formulation of Chinese herbal medicines under the best condition to elevate the yield of mycelium and polysaccharides and to promote the anti-tumor activity. The object is solved according to the features described in the claim 4.

### BRIEF DESCRIPTION OF THE DRAWING

FIG. 1 is a flow chart of fermentation process of the Phellinus linteus fungus according to the invention.

FIG. 2 is a flow chart of co-fermentation process of the Phellinus linteus fungus with Chinese herbal medicines according to the invention.

FIG. 3 is the profile of the biomass of mycelia in the fermentation of the Phellinus linteus fungus alone under the condition for the best yield control.

FIG. 4 is the profile of the yield of polysaccharides in the fermentation of the Phellinus linteus fungus alone under the condition for the best yield control.

FIG. 5 is the table and profile of the biomass of mycelia in the co-fermentation according to the invention of the Phellinus linteus fungus with Taraxacum officinale.

FIG. 6 is the table and profile of the yield of polysaccharides in the co-fermentation according to the invention of the Phellinus linteus fungus with Taraxacum officinale.

FIG. 7 is the table and profile of the biomass of mycelia in the co-fermentation according to the invention of the Phellinus linteus fungus with Lycium.

FIG. 8 is the table and profile of the yield of polysaccharides in the co-fermentation according to the invention of the Phellinus linteus fungus with Lycium.

FIG. 9 is the table and profile of the biomass of mycelia in the co-fermentation according to the invention of the Phellinus linteus fungus with a multiple-component formulation of Chinese herbal medicines.

FIG. 10 is the table and profile of the yield of polysaccharides in the co-fermentation according to the invention of the Phellinus linteus fungus with a multiple-component formulation of Chinese herbal medicines.

FIG. 11 is the table and histogram of the MTT assay on human colon cancer cell line (SW 480) treated with co-fermented medium according to the invention of the Phellinus linteus fungus with Gynostemma pentaphyllum.

FIG. 12 is the table and histogram of the MTT assay on human skin cancer cell line (A 431) treated with co-fermented medium according to the invention of the Phellinus linteus fungus with Astragalus membranaceus.

FIG. 13 is the table and histogram of the MTT assay on human prostate cancer cell line (PC-3) treated with co-fermented medium according to the invention of the Phellinus linteus fungus with Crataegus pinnatifida.

FIG. 14 is the table and histogram of the MTT assay on human hepatoma cell line (HepaG2) treated with co-fermented medium according to the invention of the Phellinus linteus fungus with Eleutherococcus senticosus.

FIG. 15 is the table and histogram of the MTT assay on human colon cancer cell line (SW 480) treated with co-fermented medium according to the invention of the Phellinus linteus fungus with Chinese herbal medicines.

FIG. 16 is the table and histogram of the MTT assay on human skin cancer cell line (A 431) treated with co-fermented medium according to the invention of the Phellinus linteus fungus with Chinese herbal medicines.

FIG. 17 is the table and histogram of the MTT assay on human prostate cancer cell line (PC-3) treated with co-fermented medium according to the invention of the Phellinus linteus fungus with Chinese herbal medicines.

FIG. 18 is the table and histogram of the MTT assay on human hepatoma cell line (HepaG2) treated with co-fermented medium according to the invention of the Phellinus linteus fungus with Chinese herbal medicines.

FIG. 19 is the table and profile of the biomass of mycelia in the co-fermentation according to the invention of the Phellinus linteus fungus with Chinese herbal medicines, where the co-fermentation is carried out by liquid fermentation in an large-scale shaker.

### DETAILED DESCRIPTION OF THE INVENTION

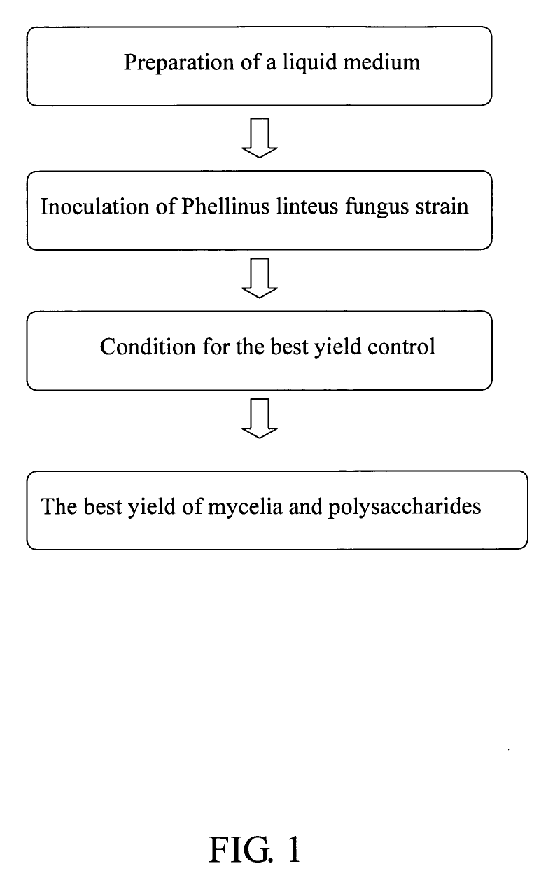

The Phellinus linteus fermentation process according to the invention (see FIG. 1) is carried out with a liquid medium (1) and a strain of the Phellinus linteus (2) fungus under the condition for the best yield control with the aim to generate the best yield of mycelia and polysaccharides, in which the liquid medium (1) consists of potato extract and glucose and the best ratio thereof is 200 g of the potato extract and 20 g of glucose per liter.

The Phellinus linteus (2) is a precious medicinal mushroom and possesses the anti-tumor and the immunity-promoting activities. The fungus is inoculated to the liquid medium (1) and incubated under the condition for the best yield control (3).

The condition for the best yield control (3) includes 4% (w/v) of glucose, 1% (w/v) of corn steep powder, 0.1% (w/v) of CaCl₂, environmental pH 4 and 125 rpm (rotation per minute) that can control the best yield of mycelia and polysaccharides.

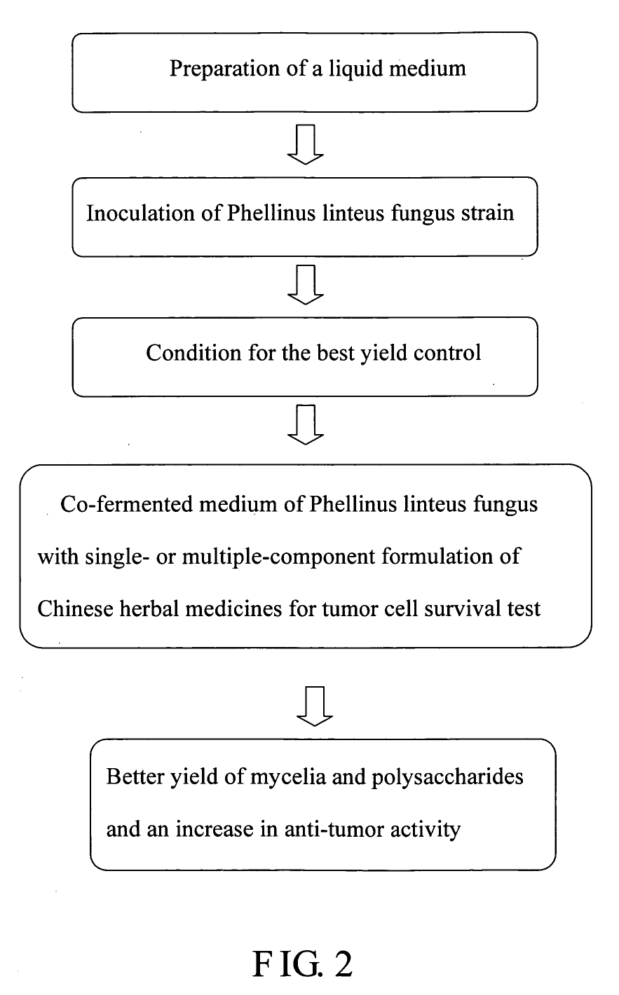

The co-fermentation of the Phellinus linteus fungus with Chinese herbal medicines according to the invention (see FIG. 2) is carried out, in which the Phellinus linteus (2) is inoculated to the liquid medium (1) and co-fermented with a single- or multiple-component formulation of Chinese herbal medicines (4) under the condition for the best yield control, in which the liquid medium (1) consists of potato extract and glucose and the best ratio thereof is 200 g of the potato extract and 20 g of glucose per liter.

The Phellinus linteus (2) is a precious medicinal mushroom and possesses the anti-tumor and the immunity-promoting activities. The fungus was inoculated to the liquid medium (1) and incubated under the condition for the best yield control (3).

The condition for the best yield control (3) includes 4% (w/v) of glucose, 1% (w/v) of corn steep powder, 0.1% (w/v) of CaCl₂, environmental pH 4 and 125 rpm (rotation per minute) that can control the best yield of mycelium and polysaccharides.

Chinese herbal medicines (4) are used by extracting the substances from the herbs. When a formulation comprises only one kind of Chinese herbal medicine (4), it is called single-component formulation. When a formulation consists of more than one kind of Chinese herbal medicine (4), it is then called multiple-component formulation. The Phellinus linteus mushroom (2) is co-fermented with a single- or multiple-component formulation of Chinese herbal medicines (4) under condition for the best yield control (3). The result shows the co-fermentation leads to a higher yield of mycelia and polysaccharides and an increase in the anti-tumor activity, compared to the fermentation of the Phellinus linteus (2) fungus alone.

### Embodiment of the Invention

The first thing is to find out the condition for the best yield control (3), for example the most suitable carbon and nitrogen sources, inorganic salts, pH value and rotation speed. The carbon source can be taken up and metabolized by the mushroom to supply the energy. The nitrogen source and pH value influence the yield of mycelium and polysaccharides. The yield of the polysaccharides needs to reach over 6 g/l and the total weight of the mycelia must be over 10 g/l, it then favors the production in the biotechnological industry. Subsequently, nine kinds of Chinese herbal medicines (4) (Codonopsis pilosula, Schisandra chinensis, Lycium barbarum, Crataegus pinnatifida, Astragalus membranaceus, Taraxacum officinale, Rose, Gynostemma pentaphyllum, Eleutherococcus senticosus) were selected. To find out the yield of mycelia and polysaccharides higher than that of the fermentation of the Phellinus linteus fungus alone, the fungus is then co-fermented with a single- or multiple-component formulation of Chinese herbal medicines (4) under the condition for the best yield control of the Phellinus linteus. Additionally, a MTT assay is performed on four human tumor cell lines such as human colon cancer cell line (SW 480), human skin cancer cell line (A 431), human prostate cancer cell line (PC-3) and human hepatoma cell line (Hepa G2) in order to compare the difference in the anti-cancer activity between the fermentation of the Phellinus linteus (2) fungus alone and the co-fermentation of the Phellinus linteus (2) fungus with Chinese herbal medicines (4).

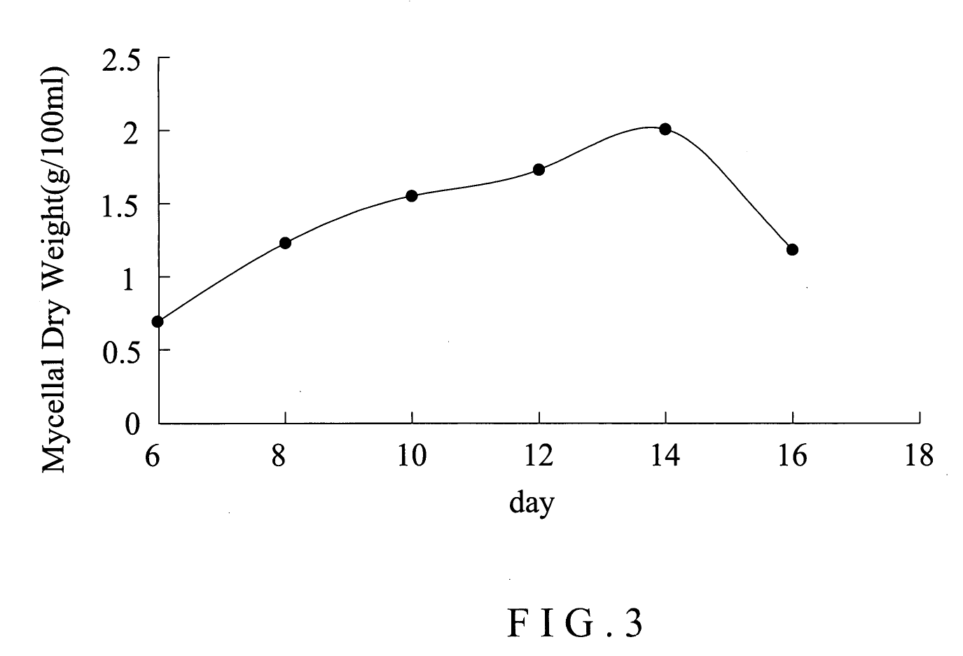

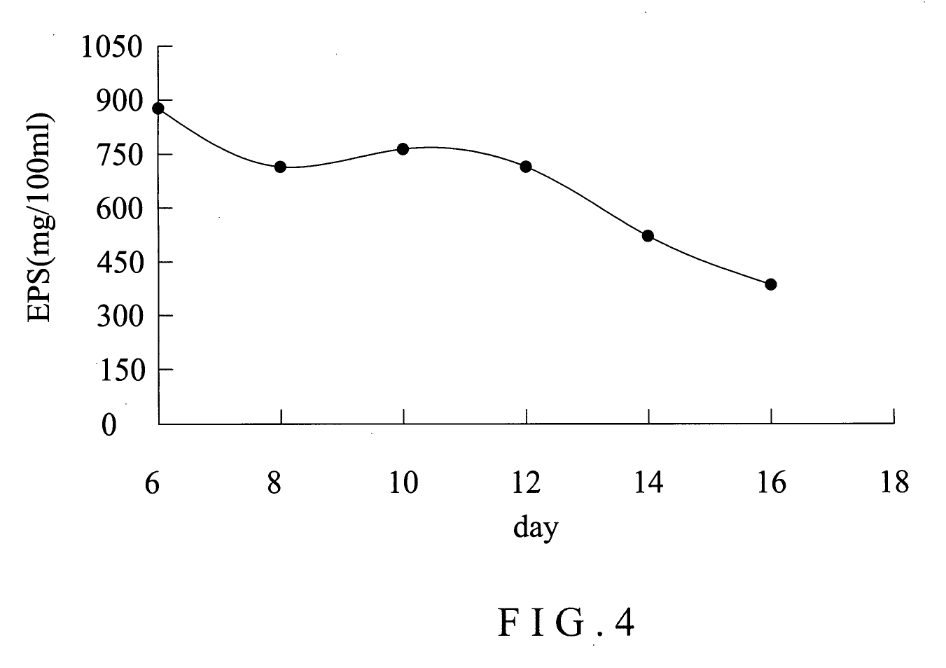

The Phellinus linteus (2) fungus was inoculated to a liquid medium and grown to 80% of confluence. The liquid medium (1) was firstly added to 250-ml sterile flasks (see FIG. 1) and the Phellinus linteus (2) fungus was then inoculated into each flask. Subsequently, 4% (w/v) of glucose as carbon source, 1% (w/v) of corn steep powder as nitrogen source and 0.1% (w/v) of CaCl₂ as inorganic salt used as condition for the best yield control (3) was added to the liquid medium (1). The mushroom was incubated at pH 4 and 125 rpm. The total weights of mycelia of the Phellinus linteus (2) fungus on days 6, 8, 10, 12 and 14 were observed (see FIG. 3) and the results showed approximately 7 g/l, 12 g/l, 16 g/l, 17 g/l and 20 g/l respectively. The total weight of mycelia obviously reached to the level of more than 10 g/l from day 8 on. The polysaccharide yield of the Phellinus linteus (2) fungus on days 6, 8, 10, 12 and 14 were observed (see FIG. 4) and the results showed approximately 9 g/l, 7 g/l, 7.5 g/l, 7 g/l and 5.5 g/l respectively. The polysaccharide yield could reach to the level of more than 6 g/l from day 6 to day 12.

Nine Chinese herbal medicines (4) (Codonopsis pilosula, Schisandra chinensis, Lycium barbarum, Crataegus pinnatifida, Astragalus membranaceus, Taraxacum officinale, Rose, Gynostemma pentaphyllum, Eleutherococcus senticosus) were individually boiled in water, concentrated and dried at 60° C. The crude extracts thereof (4) were thus obtained. The experiments on the co-fermentation of the Phellinus linteus (2) fungus with a single- or multiple-component formulation of Chinese herbal medicines (4) were carried out under the condition for the best yield control (3) (see FIG. 2).

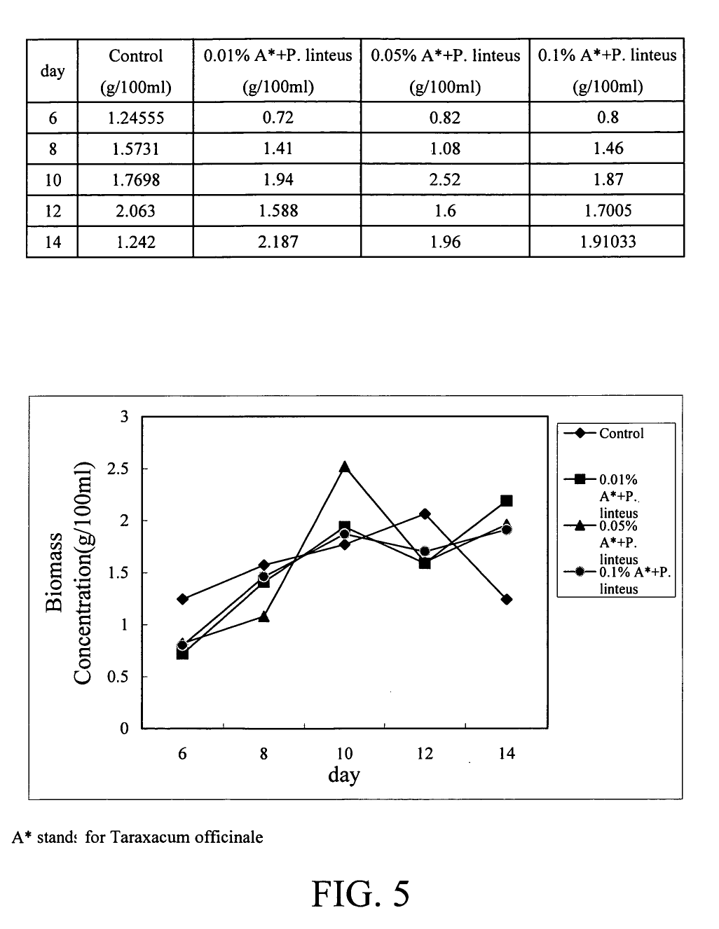

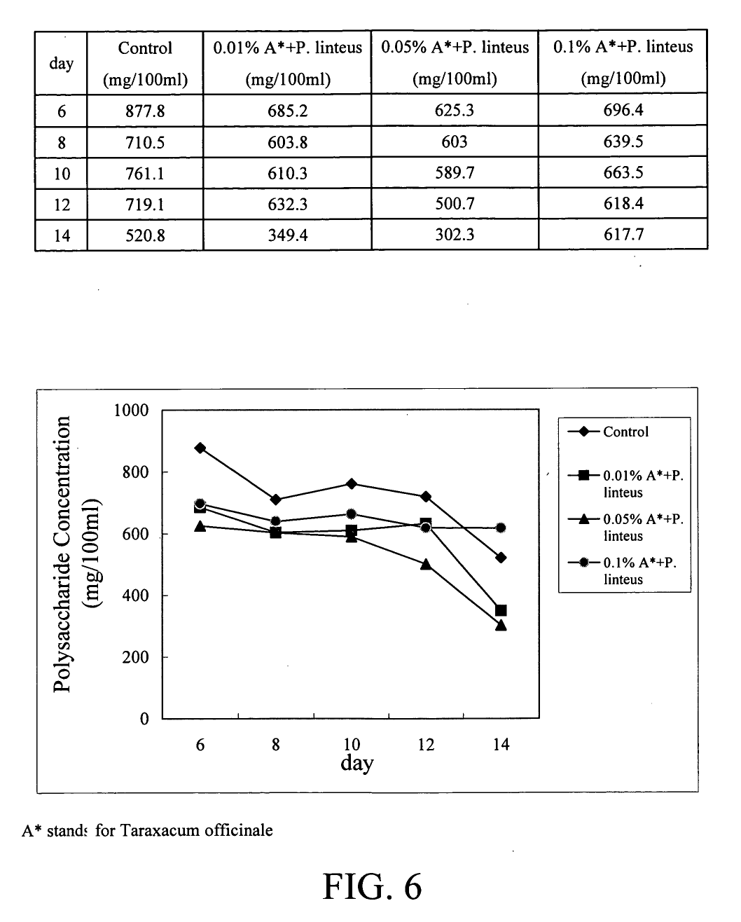

### Experiment 1: Co-Fermentation of the Phellinus linteus (2) Fungus with a Single-Component Formulation of Chinese Herbal Medicine (4), Taraxacum officinale (see FIGS. 5 and 6)

A single-component formulation of Chinese herbal medicine (4), Taraxacum officinale, was added to the Phellinus linteus (2) fungus and co-fermented under the condition for the best yield control (3), while the fermentation of the Phellinus linteus (2) fungus alone under the condition for the best yield control (3) was used as control. Three different concentrations of Taraxacum officinale, 0.01%, 0.05% and 0.1% (w/v), were used in the experiment and the yield of mycelia and polysaccharides on days 6, 8, 10, 12 and 14 were observed. The result showed the yield of mycelia in groups of three concentrations, 0.01%, 0.05% and 0.1%, could be kept at more than 10 g/l after the day 8 (see FIG. 5). However, the yield in two of the concentrations of Taraxacum officinale, 0.01% and 0.05%, are actually better than that of the control group. In addition, the result showed the yield of polysaccharides in groups of three concentrations, 0.01%, 0.05% and 0.1%, could be kept at more than 6 g/l (see FIG. 6).

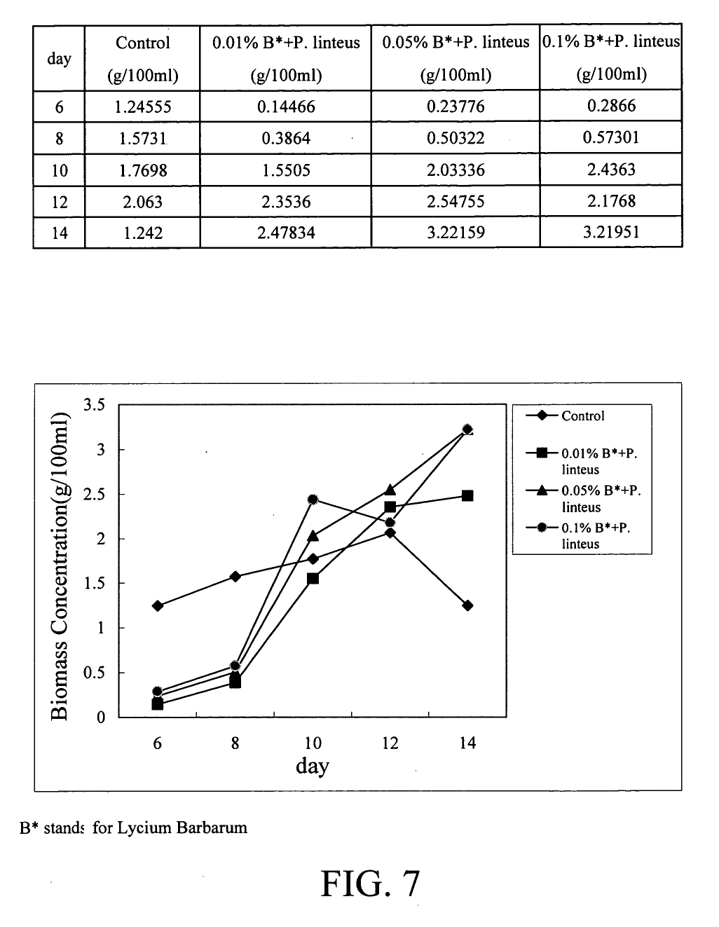

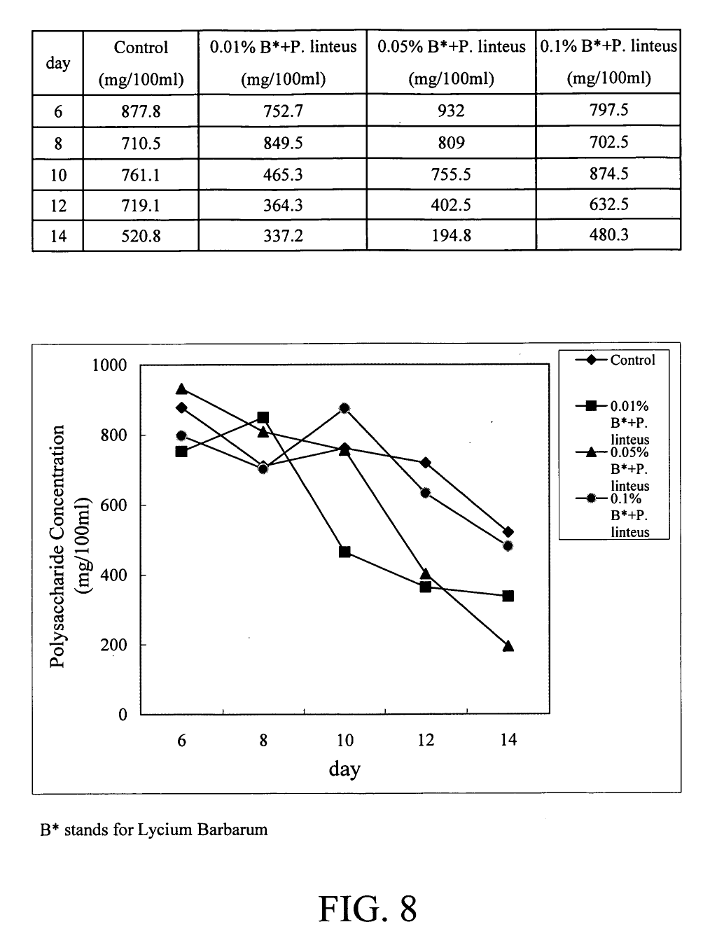

### Experiment 2: Co-Fermentation of the Phellinus linteus (2) Fungus with a Single-Component Formulation of Chinese Herbal Medicine (4), Lycium barbarum (see FIGS. 7 and 8)

A single-component formulation of Chinese herbal medicine (4), Lycium barbarum was added to the Phellinus linteus (2) fungus and co-fermented under the condition for the best yield control (3), while the fermentation of the Phellinus linteus (2) fungus alone under the condition for the best yield control (3) was used as control. Three different concentrations of Lycium barbarum, 0.01%, 0.05% and 0.1% (w/v), were used in the experiment and the yield of mycelia and polysaccharides on days 6, 8, 10, 12 and 14 were observed. The result showed the yield of mycelia in groups of three concentrations, 0.01%, 0.05% and 0.1%, could be kept at more than 10 g/l after the day 10 (see FIG. 7). The yield in all of the three concentrations of Lycium barbarum, 0.01%, 0.05% and 0.1%, was better than that of the control group from day 10 to day 14. In addition, the result showed the yield of polysaccharides in two of the concentrations of Lycium barbarum, 0.05% and 0.1%, was better than that of the control group from day 6 to day 10 (see FIG. 8). The yield of polysaccharides in groups of three concentrations, 0.01%, 0.05% and 0.1%, was more than 6 g/l from day 6 to day 10.

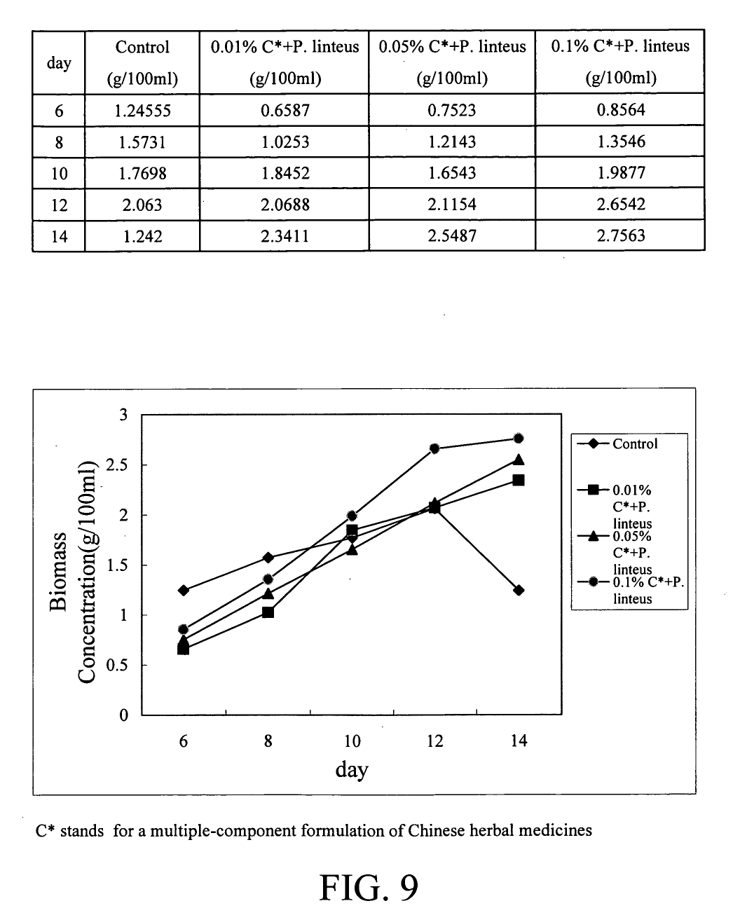

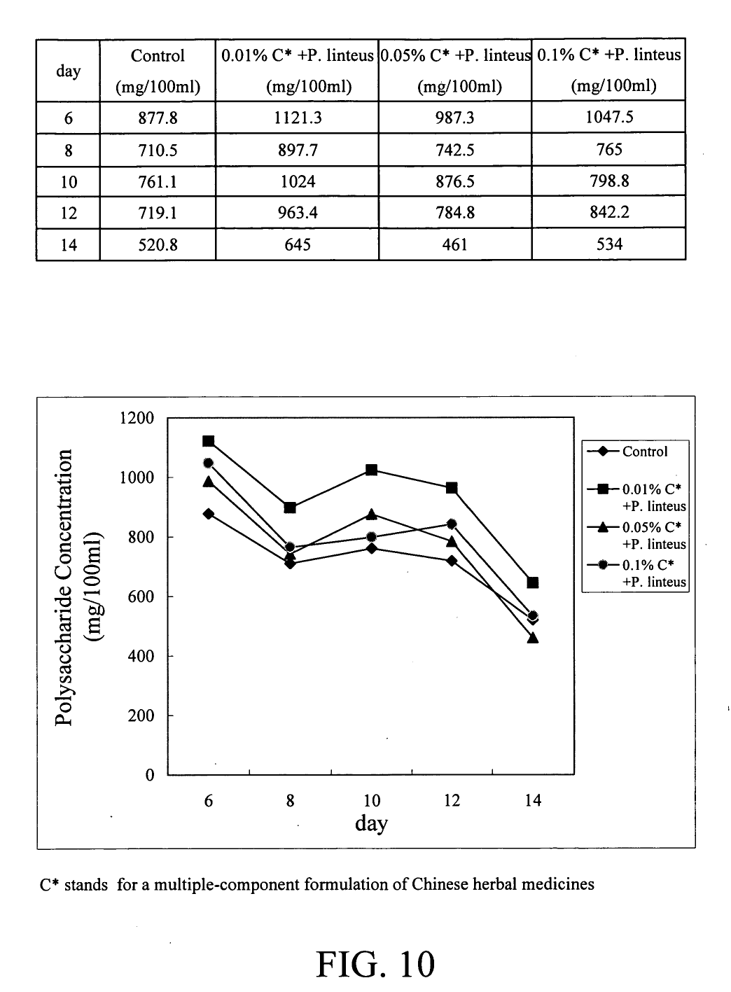

### Experiment 3: Co-Fermentation of the Phellinus linteus (2) Fungus with a Multiple-Component Formulation of Chinese Herbal Medicines (4), Rose, Astragalus membranaceus, Schisandra chinensis, Lycium barbarum and Codonopsis pilosula (see FIGS. 9 and 10)

A multiple-component formulation of Chinese herbal medicines (4), Rose, Astragalus membranaceus, Schisandra chinensis, Lycium barbarum and Codonopsis pilosula, was added to the Phellinus linteus (2) fungus and co-fermented under the condition for the best yield control (3), while the fermentation of the Phellinus linteus (2) fungus alone under the condition for the best yield control (3) was used as control. Three different concentrations of the formulation of Chinese herbal medicines (4), 0.01%, 0.05% and 0.1% (w/v), were used in the experiment and the yield of mycelia and polysaccharides on days 6, 8, 10, 12 and 14 were observed. The result showed the yield of mycelia in groups of three concentrations, 0.01%, 0.05% and 0.1%, could be kept at more than 10 g/l after the day 8 (see FIG. 9). The yield in two of the concentrations of Chinese herbal medicines (4), 0.01% and 0.1%, was better than that of the control group. In addition, the result showed the yield of polysaccharides in groups of the three concentrations of Chinese herbal medicines (4), 0.01%, 0.05% and 0.1%, was better than that of the control group (see FIG. 10). The yield of polysaccharides in groups of three concentrations, 0.01%, 0.05% and 0.1%, was more than 8 g/l.

### Tumor Cell Survival Test (MTT Assay) (see FIG. 2)

To compare the difference in the anti-tumor activity between the fermentation of the Phellinus linteus (2) fungus alone and the co-fermentation of the Phellinus linteus (2) fungus with Chinese herbal medicines (4), four human tumor cell lines were respectively transferred into 24-well Petri dishes and incubated at 37° C. and 5% CO₂ for 24 hours.

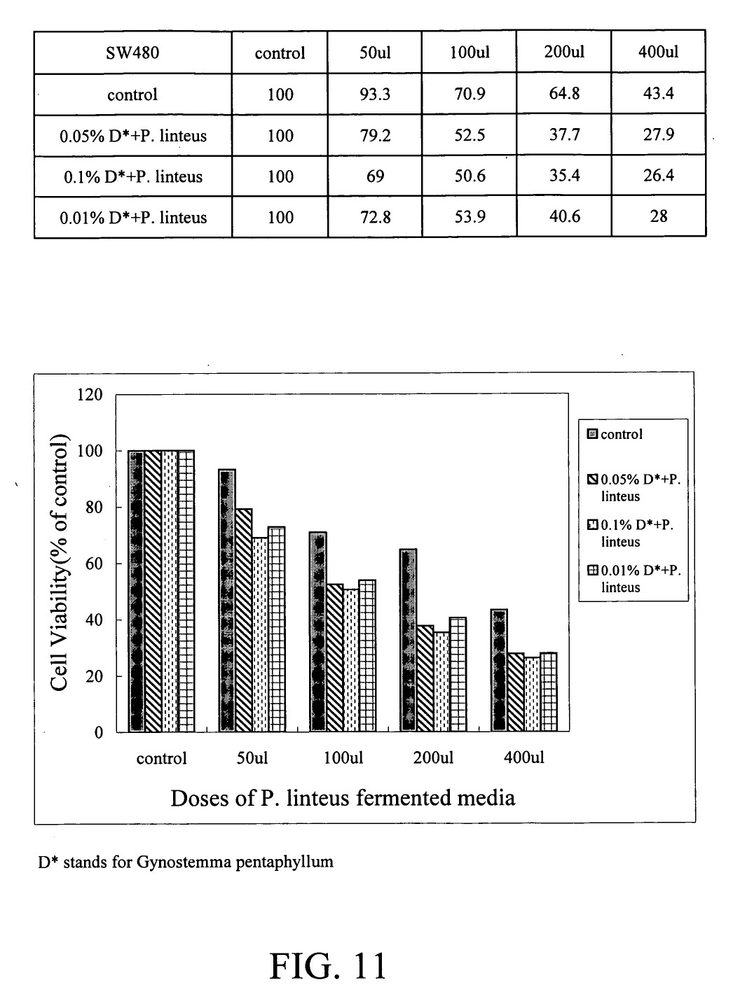

### Experiment 4: Anti-Tumor Effect of the Co-Fermented Medium of the Phellinus linteus (2) Fungus with Chinese Herbal Medicine (4), Gynostemma pentaphyllum, on the Human Colon Cancer Cell Line (SW 480) (see FIG. 11)

Each of the co-fermented media of the Phellinus linteus (2) fungus with three concentrations (0.01%, 0.05% and 0.1%, w/v) of Chinese herbal medicine (4), Gynostemma pentaphyllum, was added to the human colon cancer cell line (SW 480) in four different doses, 50 μl, 100 μl, 200 μl and 400 μl, while a treatment of the same cancer cells with four different doses (50 μl, 100 μl, 200 μl and 400 μl) of the fermented medium of the Phellinus linteus fungus alone was used as control. After incubation at 37° C. and 5% CO₂ for 72 hours, the medium was removed and washed with PBS. The cells were then fixed by ethanol and stained by crystal violet. Subsequently, the tumor cell survival rates were determined at 590 μm. The results showed inhibitory effect on the human colon cancer cell line (SW 480) in groups of the co-fermented media of the Phellinus linteus (2) fungus with three concentrations of Gynostemma pentaphyllum was better than that of .the fermented medium of the Phellinus linteus (2) fungus alone. In particular, when the dose increased up to more than 100 μl, the cancer cells could be killed and the survival rate thereof fell to less than 60%. This result significantly showed an anti-tumor effect.

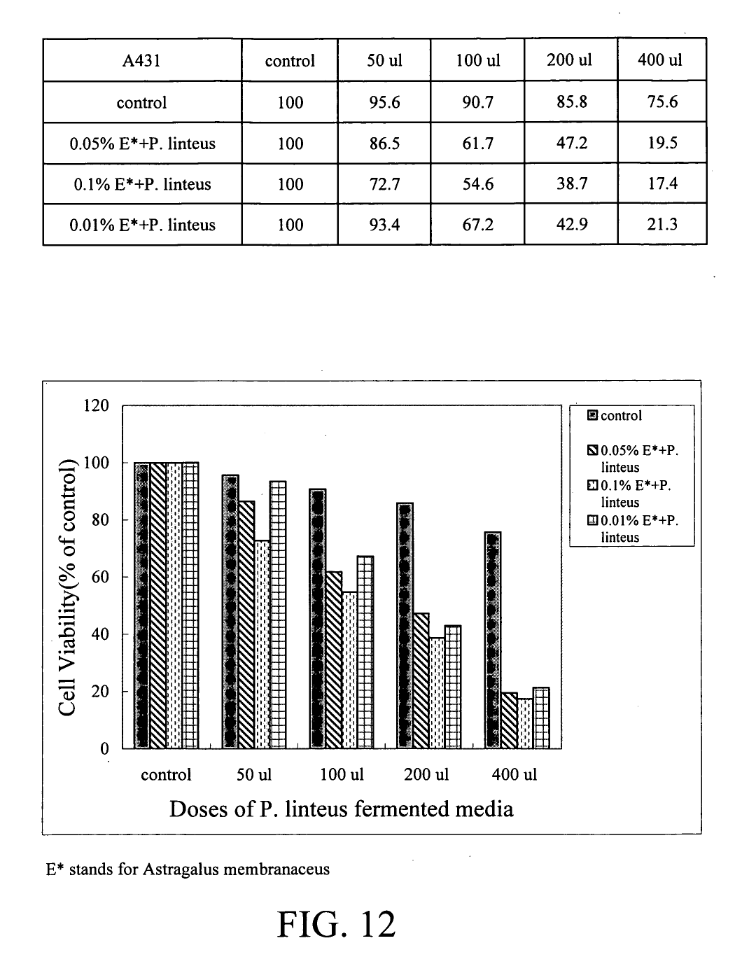

### Experiment 5: Anti-Tumor Effect of the Co-Fermented Medium of the Phellinus linteus (2) Fungus with Chinese Herbal Medicine (4), Astragalus membranaceus, on the Human Skin Cancer Cell Line (A 431) (see FIG. 12)

Each of the co-fermented media of the Phellinus linteus (2) fungus with three concentrations (0.01%, 0.05% and 0.1%, w/v) of Chinese herbal medicine (4), Astragalus membranaceus, was added to the human skin cancer cell line (A 431) in four different doses, 50 μl, 100 μl, 200 μl and 400 μl, while a treatment of the same cancer cells with four different doses (50 μl, 100 μl, 200 μl and 400 μl) of .the fermented medium of the Phellinus linteus (2) fungus alone was used as control. The results showed inhibitory effect on the human skin cancer cell line (A 431) in groups of the co-fermented media of the Phellinus linteus (2) fungus with three concentrations of Astragalus membranaceus was better than that of the fermented medium of the Phellinus linteus (2) fungus alone. In particular, when the dose was up to 100 μl, the survival rate of the cancer cells fell to less than 60%. When the volume was further up to 400 μl, the survival rate decreased to less than 20%.

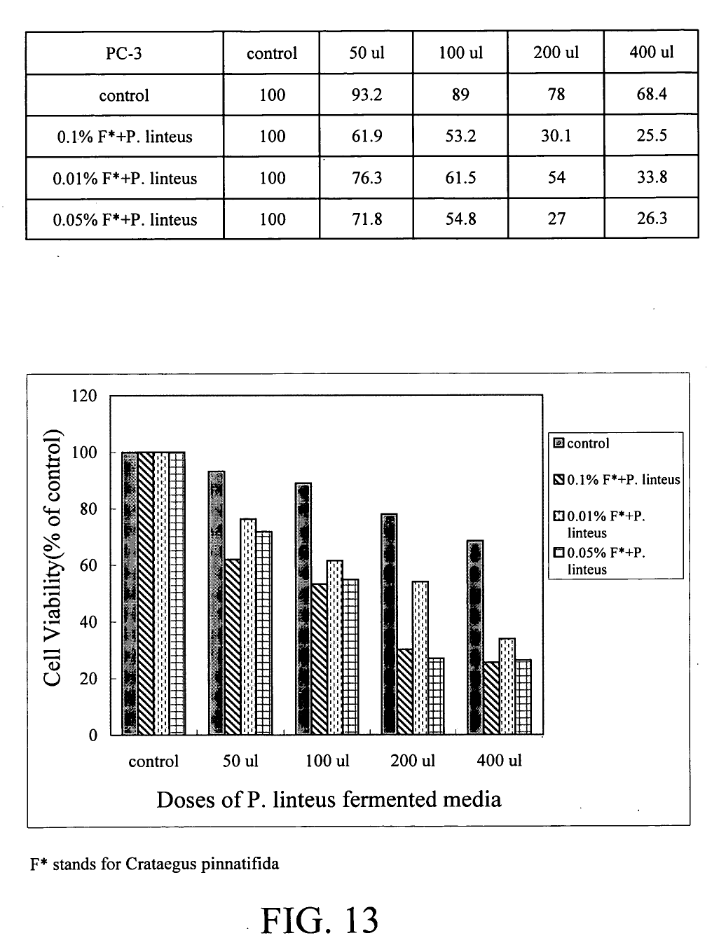

### Experiment 6: Anti-Tumor Effect of the Co-Fermented Medium of the Phellinus linteus (2) Fungus with Chinese Herbal Medicine (4), Crataegus pinnatifida, on the Human Prostate Cancer Cell Line (PC-3) (see FIG. 13)

Each of the co-fermented media of the Phellinus linteus (2) fungus with three concentrations (0.01%, 0.05% and 0.1%, w/v) of Chinese herbal medicine (4), Crataegus pinnatifida, was added to the human prostate cancer cell line (PC-3) in four different doses, 50 μl, 100 μl, 200 μl and 400 μl, while a treatment of the same cancer cells with four different doses (50 μl, 100 μl, 200 μl and 400 μl) of the fermented medium of the Phellinus linteus (2) fungus alone was used as control. The results showed inhibitory effect on the human prostate cancer cell line (PC-3) in groups of the co-fermented media of the Phellinus linteus (2) fungus with three concentrations of Crataegus pinnatifida was better than that of the fermented medium of the Phellinus linteus (2) fungus alone. In particular, when the dose was up to 100 μl, the survival rate of the cancer cells fell to less than 60%. The effect would be better when the dose increased.

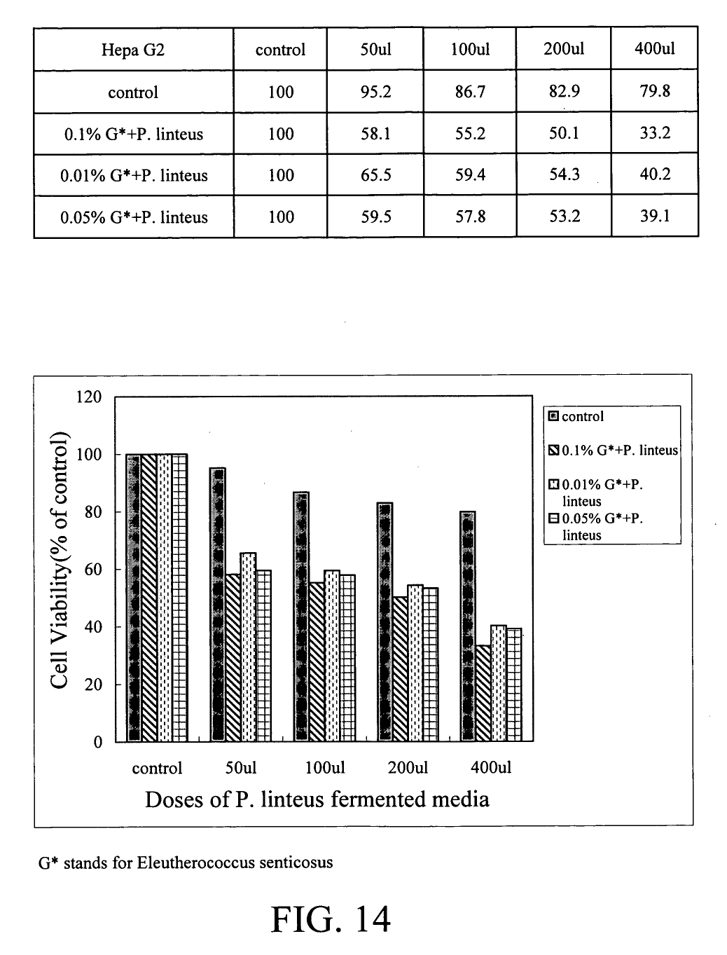

### Experiment 7: Anti-Tumor Effect of the Co-Fermented Medium of the Phellinus linteus (2) Fungus with Chinese Herbal Medicine (4), Eleutherococcus senticosus, on the Human Hepatoma Cell Line (Hepa G2) (see FIG. 14)

Each of the co-fermented media of the Phellinus linteus (2) fungus with three concentrations (0.01%, 0.05% and 0.1%, w/v) of Chinese herbal medicine (4), Eleutherococcus senticosus, was added to the human hepatoma cell line (Hepa G2) in four different doses, 50 μl, 100 μl, 200 μl and 400 μl, while a treatment of the same cancer cells with four different doses (50 μl, 100 μl, 200 μl and 400 μl) of the fermented medium of the Phellinus linteus (2) fungus alone was used as control. The results showed inhibitory effect on the human prostate cancer cell line (PC-3) in groups of the co-fermented media of the Phellinus linteus (2) fungus with three concentrations of Eleutherococcus senticosus was better than that of .the fermented medium of the Phellinus linteus (2) fungus alone. In particular, when 50 μl were used, the survival rate of the cancer cells fell to less than 60%. The effect would be better when the dose increased.

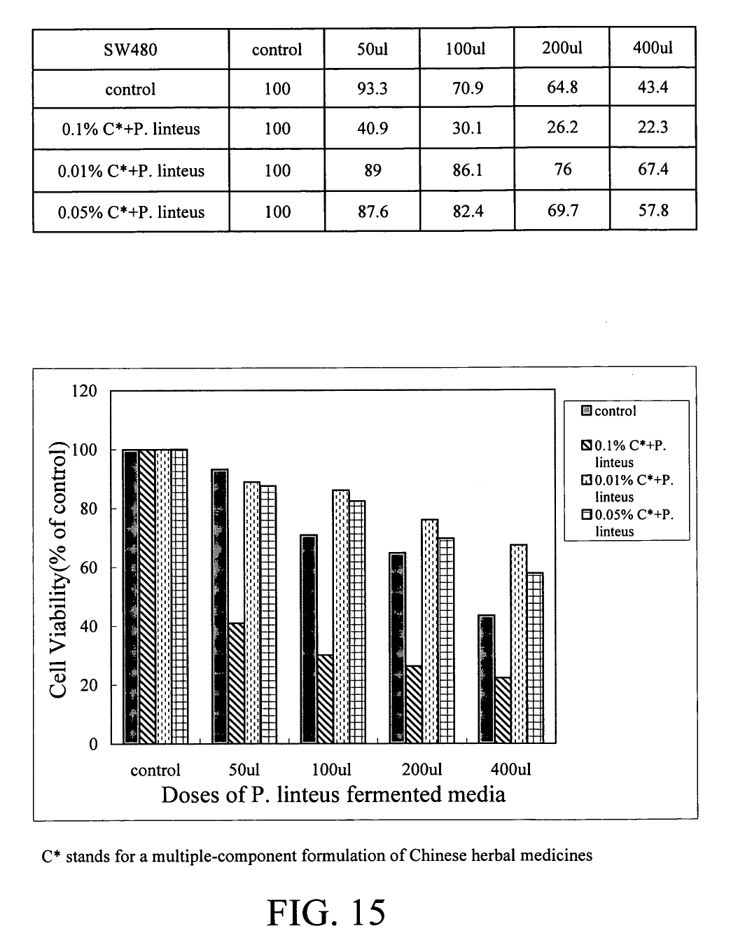

Experiment 8: Anti-Tumor Effect of the Co-Fermented Medium of the Phellinus linteus (2) Fungus with Chinese Herbal Medicines (4), Rose, Astragalus membranaceus, Schisandra chinensis, Lycium barbarum and Codonopsis pilosula, on the Human Colon Cancer Cell Line (SW 480) (see FIG. 15)

Each of the co-fermented media of the Phellinus linteus (2) fungus with three concentrations (0.01%, 0.05% and 0.1%, w/v) of Chinese herbal medicines (4), Rose, Astragalus membranaceus, Schisandra chinensis, Lycium barbarum and Codonopsis pilosula, was added to the human colon cancer cell line (SW 480) in four different doses, 50 μl, 100 μl, 200 μl and 400 μl, while a treatment of the same cancer cells with four different doses (50 μl, 100 μl, 200 μl and 400 μl) of .the fermented medium of the Phellinus linteus (2) fungus alone was used as control. The results showed inhibitory effect on the human colon cancer cell line (SW 480) in the co-fermented medium of the Phellinus linteus (2) with 0.1% of Chinese herbal medicines (4), Rose, Astragalus membranaceus, Schisandra chinensis, Lycium barbarum and Codonopsis pilosula, was better than that of the fermented medium of the Phellinus linteus (2) alone. When 50 μl of co-fermented medium of the Phellinus linteus (2) with 0.1% of Chinese herbal medicines (4) were used, the survival rate of the cancer cells fell to less than 60%. The effect would be better when the dose increased.

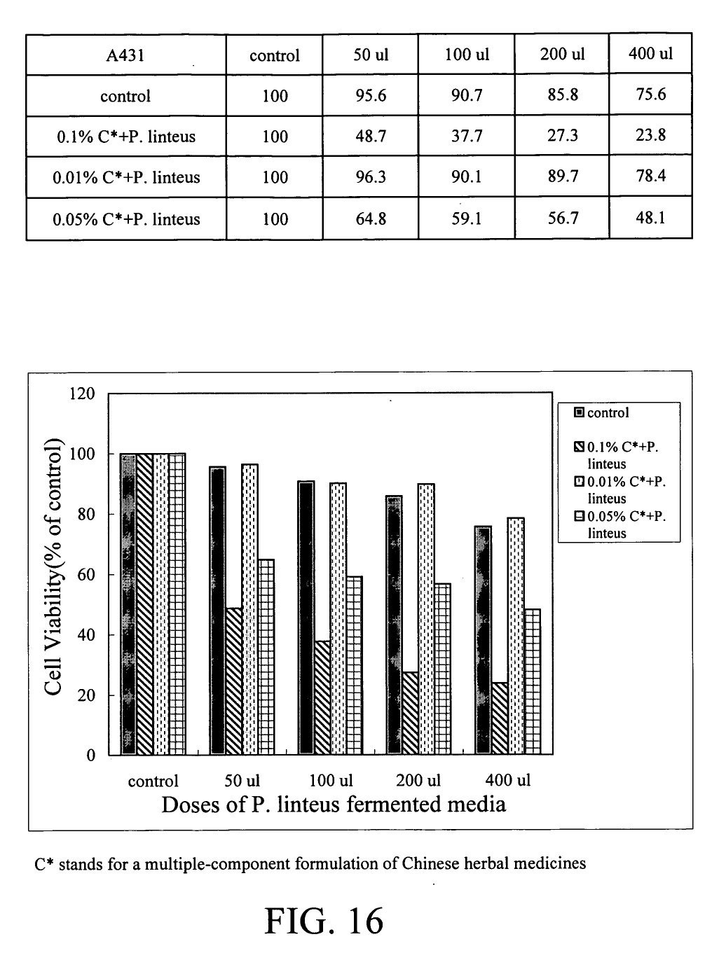

Experiment 9: Anti-Tumor Effect of the Co-Fermented Medium of the Phellinus linteus (2) Fungus with Chinese Herbal Medicines (4), Rose, Astragalus membranaceus, Schisandra chinensis, Lycium barbarum and Codonopsis pilosula, on the Human Skin Cancer Cell Line (A 431) (see FIG. 16)

Each of the co-fermented media of the Phellinus linteus (2) fungus with three concentrations (0.01%, 0.05% and 0.1%, w/v) of Chinese herbal medicines (4), Rose, Astragalus membranaceus, Schisandra chinensis, Lycium barbarum and Codonopsis pilosula, was added to the human skin cancer cell line (A 431) in four different doses, 50 μl, 100 μl, 200 μl and 400 μl, while a treatment of the same cancer cells with four different doses (50 μl, 100 μl, 200 μl and 400 μl) of .the fermented medium of the Phellinus linteus (2) fungus alone was used as control. The results showed inhibitory effect on the human skin cancer cell line (A 431) in the co-fermented media of the Phellinus linteus (2) fungus with 0.05% and 0.1% of Chinese herbal medicines (4), Rose, Astragalus membranaceus, Schisandra chinensis, Lycium barbarum and Codonopsis pilosula, was better than that of the fermented medium of the Phellinus linteus (2) fungus alone. When 50 μl of co-fermented medium of the Phellinus linteus (2) fungus with 0.1% of Chinese herbal medicines (4) were used, the survival rate of the cancer cells fell to less than 60%. The effect would be better when the dose increased.

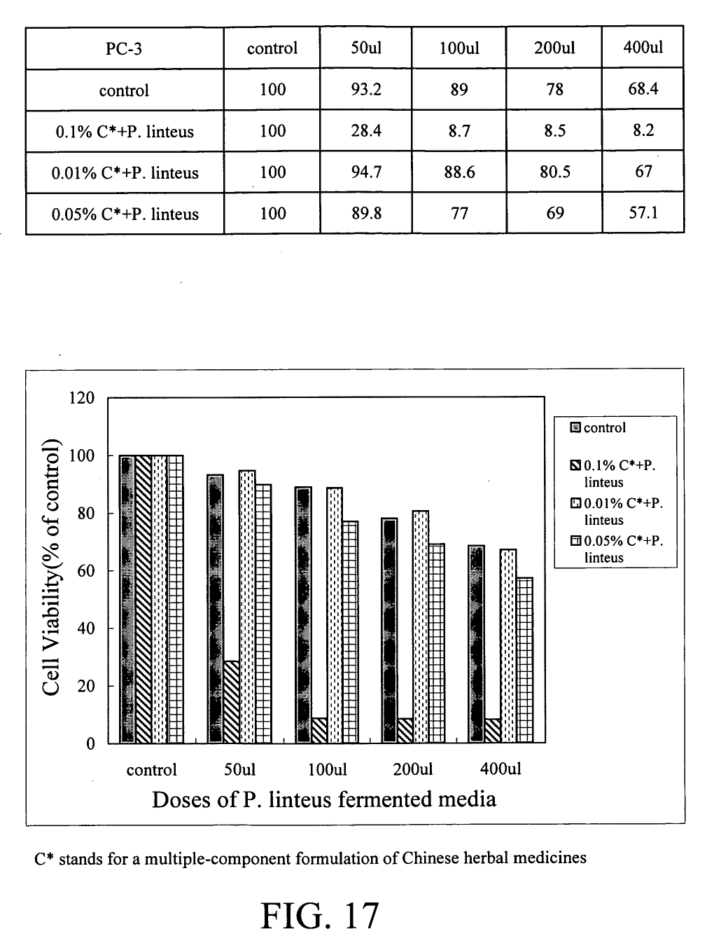

Experiment 10: Anti-Tumor Effect of the Co-Fermented Medium of the Phellinus linteus (2) Fungus with Chinese Herbal Medicines (4), Rose, Astragalus membranaceus, Schisandra chinensis, Lycium barbarum and Codonopsis pilosula, on the Human Prostate Cancer Cell Line (PC-3) (see FIG. 17)

Each of the co-fermented media of the Phellinus linteus (2) fungus with three concentrations (0.01%, 0.05% and 0.1%, w/v) of Chinese herbal medicines (4), Rose, Astragalus membranaceus, Schisandra chinensis, Lycium barbarum and Codonopsis pilosula, was added to the human prostate cancer cell line (PC-3) in four different dosees, 50 μl, 100 μl, 200 μl and 400 μl, while a treatment of the same cancer cells with four different doses (50 μl, 100 μl, 200 μl and 400 μl) of the fermented medium of the Phellinus linteus (2) fungus alone was used as control. The results showed inhibitory effect on the human prostate cancer cell line (PC-3) in the co-fermented medium of the Phellinus linteus (2) fungus with 0.1% of Chinese herbal medicines (4), Rose, Astragalus membranaceus, Schisandra chinensis, Lycium barbarum and Codonopsis pilosula, was better than that of the fermented medium of the Phellinus linteus (2) fungus alone. When 50 μl of co-fermented medium of the Phellinus linteus (2) fungus with 0.1% of Chinese herbal medicines (4) were used, the survival rate of the cancer cells fell to less than 30%. When the dose increased up to 100 ml, the survival rate decreased to less than 9%. The inhibitory effect was very excellent.

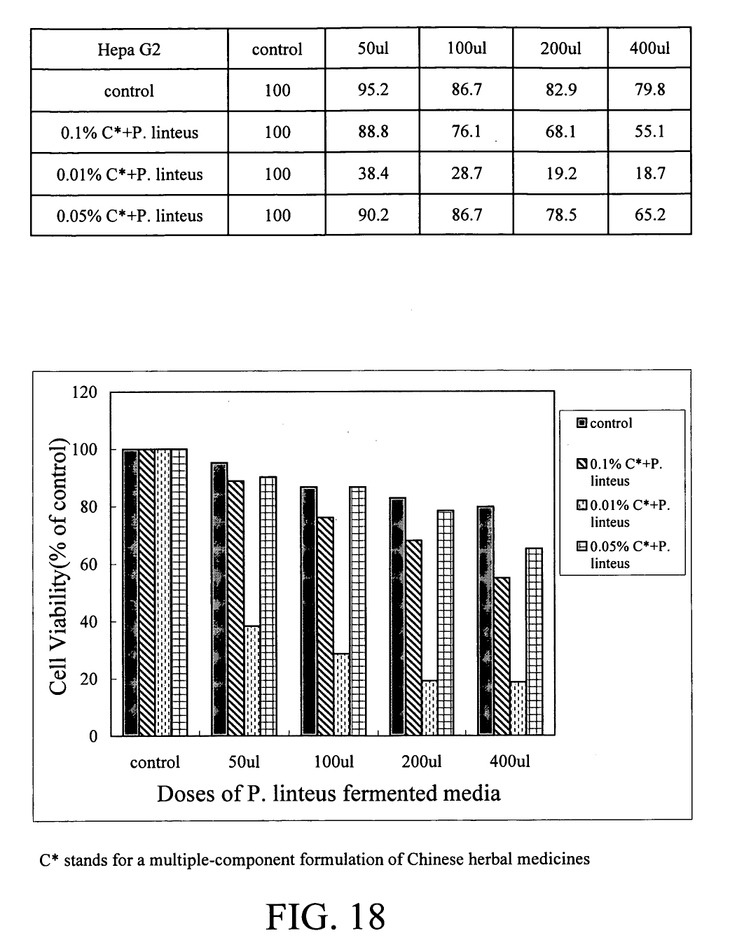

Experiment 11: Anti-Tumor Effect of the Co-Fermented Medium of the Phellinus linteus (2) Fungus with Chinese Herbal Medicines (4), Rose, Astragalus membranaceus, Schisandra chinensis, Lycium barbarum and Codonopsis pilosula, on the Human Hepatoma Cell Line (Hepa G2) (see FIG. 18)

Each of the co-fermented media of the Phellinus linteus (2) fungus with three concentrations (0.01%, 0.05% and 0.1%, w/v) of Chinese herbal medicines (4), Rose, Astragalus membranaceus, Schisandra chinensis, Lycium barbarum and Codonopsis pilosula, was added to the human hepatoma cell line (Hepa G2) in four different doses, 50 μl, 100 μl, 200 μl and 400 μl, while a treatment of the same cancer cells with four different doses (50 μl, 100 μl, 200 μl and 400 μl) of .the fermented medium of the Phellinus linteus (2) fungus alone was used as control. The results showed inhibitory effect on the human hepatoma cell line (Hepa G2) in the co-fermented medium of the Phellinus linteus (2) fungus with 0.05% and 0.1% of Chinese herbal medicines (4), Rose, Astragalus membranaceus, Schisandra chinensis, Lycium barbarum and Codonopsis pilosula, was better than that of .the fermented medium of the Phellinus linteus (2) fungus alone. When 50 μl of co-fermented medium of the Phellinus linteus (2) fungus with 0.1% of Chinese herbal medicines (4) were used, the survival rate of the cancer cells fell to less than 40%. The effect would be better when the dose increased. The co-fermented medium of the Phellinus linteus (2) fungus with 0.1% of Chinese herbal medicines (4), Rose, Astragalus membranaceus, Schisandra chinensis, Lycium barbarum and Codonopsis pilosula caused the survival rate of the cancer cells to decrease to less than 40% and showed the best anti-cancer activity.

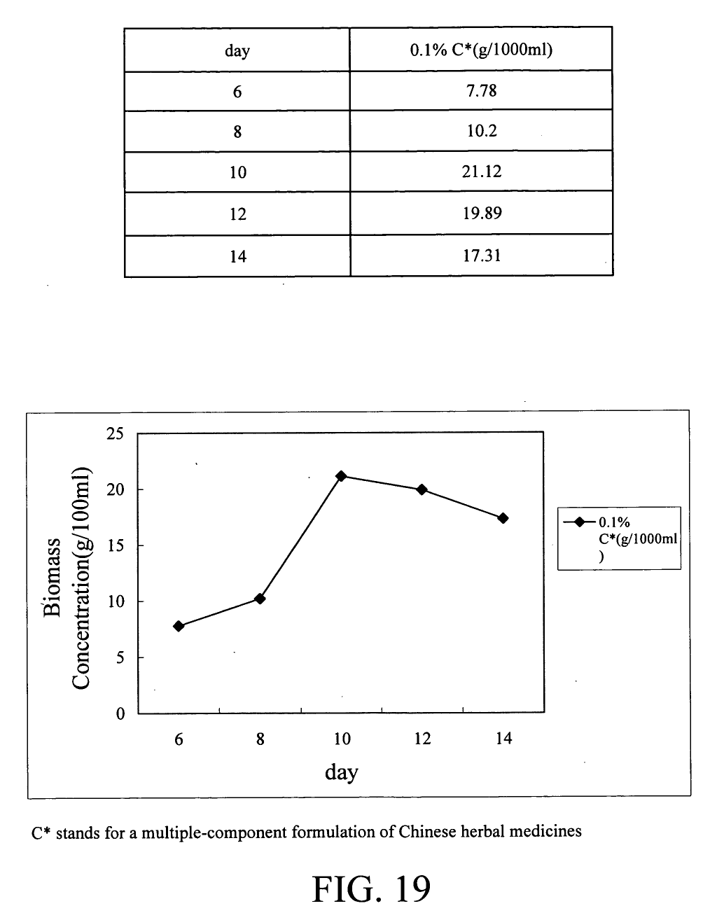

### Experiment 12: Mass Production by Liquid Fermentation in Large-Scale Shaker (see FIG. 19)

The Phellinus linteus (2) fungus was co-fermented with 0.1% (w/v) of Chinese herbal medicines (4), Rose, Astragalus membranaceus, Schisandra chinensis, Lycium barbarum and Codonopsis pilosula, in the condition: 4% (w/v) of glucose, 1% (w/v) of corn steep powder, 0.05% (w/v) of KH₂PO₄, pH 4 and 125 rpm for 6 to 14 days. The biomass of mycelia on days 6, 8, 10, 12 and 14 were observed and the resulting weights were approximately 7.78 g/l, 10.20 g/l, 21.12 g/l, 19.89 g/l and 17.31 g/l respectively. The biomass of mycelia on day 8 has reached to the level of 10 g/l. In addition, the biomass of mycelia from day 8 to day 14 could be kept at the level of more than 10 g/l, the yield of which is enough for the mass production in the biotechnological industry.

To sum up, the present invention obviously involves an inventive step and has not been disclosed in any other publication. The examples described above are simply the preferred embodiments of the invention so that they do not limit the scope of the invention.

1. A process of fermenting the fungus of Phellinus linteus, in which the liquid medium as well as the strain of Phellinus linteus are under condition for the best yield control to generate the best yield of mycelia and polysaccharides, where the liquid medium consists of potato extract and glucose and the Phellinus linteus mushroom is inoculated to the medium and incubated in the condition for the best yield control.

2. The process of fermenting the fungus of Phellinus linteus according to claim 1, wherein the best ratio of the liquid medium is 200 g of potato extract and 20 g of glucose per liter.

3. The process of fermenting the fungus of Phellinus linteus according to claim 1, wherein the condition for the best yield control includes 4% (w/v) of glucose, 1% (w/v) of corn steep powder, 0.1% (w/v) of CaCl₂, pH 4 and 125 rpm, which leads to the best yield of mycelia and polysaccharides.

4. A process of co-fermenting the fungus of Phellinus linteus with Chinese herbal medicines, in which a single- or multiple-component formulation of Chinese herbal medicines is added to a liquid medium having the fungus of Phellinus linteus and co-fermented in the condition for the best yield control, wherein the liquid medium consists of potato extract and glucose and the Phellinus linteus fungus is inoculated to the medium and co-fermented with a single- or multiple-component formulation of Chinese herbal medicines in the condition for the best yield control, which leads to a better yield of mycelia and polysaccharides and an increase in the anti-tumor activity, compared to the fermentation of the Phellinus linteus fungus alone.

5. The process of co-fermenting the fungus of Phellinus linteus with Chinese herbal medicines according to claim 4, wherein the best ratio of the liquid medium is 200 g of potato extract and 20 g of glucose per liter.

6. The process of co-fermenting the fungus of Phellinus linteus with Chinese herbal medicines according to claim 4, wherein the condition for the best yield control includes 4% (w/v) of glucose, 1% (w/v) of corn steep powder, 0.1% (w/v) of CaCl₂, pH 4 and 125 rpm, which leads to the best yield of mycelia and polysaccharides.

7. The process of co-fermenting the fungus of Phellinus linteus with Chinese herbal medicines according to claim 4, wherein the single-component formulation of Chinese herbal medicines is selected from one of the nine Chinese herbal medicines, Codonopsis pilosula, Schisandra chinensis, Lycium barbarum, Crataegus pinnatifida, Astragalus membranaceus, Taraxacum officinale, Rose, Gynostemma pentaphyllum, Eleutherococcus senticosus.

8. The process of co-fermenting the fungus of Phellinus linteus with Chinese herbal medicines according to claim 4, wherein the multiple-component formulation of Chinese herbal medicines is selected from two or more of the nine Chinese herbal medicines, Codonopsis pilosula, Schisandra chinensis, Lycium barbarum, Crataegus pinnatifida, Astragalus membranaceus, Taraxacum officinale, Rose, Gynostemma pentaphyllum, Eleutherococcus senticosus.
